(19) United States
(12) Patent Application Publication
(10) Pub. No.: US 2002/0151952 A1
(43) Pub. Date: Oct. 17, 2002

(54) NECESSARIES FOR TREATING A BLOOD VESSEL

(76) Inventor: ERIC PEROUSE, L'ISLE ADAM (FR)

Correspondence Address: WENDEROTH LIND AND PONACK LLP, 2033 K STREET NW SUITE 800, WASHINGTON, DC, 20006

(21) Appl. No.: 09/332,906

(22) Filed: Jun. 15, 1999

(30) Foreign Application Priority Data
Jun. 17, 1998 (FR) 98 07653

(51) Int. Cl. A61F 2/06; A61F 11/00

(52) U.S. Cl. 623/1.11; 606/108; 206/438

(57) ABSTRACT

The necessaries for treating a blood vessel include a tubular endoprosthesis (10) whose distal end is connected to a support (12, 112) by releasable retaining means (14) and a ferrule (16) for guiding the endoprosthesis (10) connected to the support (12, 112) for implanting it in a living organism. At least the proximal end of the endoprosthesis is initially outside the ferrule (16). The endoprosthesis (10) initially has a length sufficient to be trimmed to suit the requirements of the vessel to be treated by cutting off and discarding a surplus length of the endoprosthesis (10) at its proximal end.

The present invention concerns the necessaries for treating a blood vessel of the type including a tubular endoprosthesis whose distal end is connected to a support by releasable retaining means and a ferrule for guiding the endoprosthesis connected to the support for the purpose of implanting it into a living organism.

Necessaries of the above kind are described in the Applicant's French patent application FR-A-94-05034.

The apparatus described in the above document, in particular with reference to FIG. 6, includes a tubular endoprosthesis which is initially compressed inside an external ferrule or sheath. The apparatus is supplied with the endoprosthesis radially compressed in the external ferrule to enable it to be implanted in an artery or a vein.

The dimensional characteristics of the tubular endoprosthesis must be chosen by the surgeon to suit the size of the patient and the area in which the tubular endoprosthesis is to be implanted. In particular, the diameter of the tubular endoprosthesis and its length must correspond exactly to the characteristics of the vessel into which the endoprosthesis is to be implanted.

To meet all possible requirements, the manufacturer of apparatus as described in application 94-05034 must provide a complete range of tubular endoprostheses initially compressed in this way in an implantation ferrule.

In particular, the range must allow for all possible combinations of diameter and length to suit the vessel to be treated. The multiplicity of possible combinations greatly increases the unit cost of manufacture of each apparatus. Also, the a priori choice to be made by the surgeon, with regard to the appropriate dimensions of the tubular endoprosthesis, is a difficult one.

The aim of the invention is to provide a solution to this problem by proposing necessaries for treating a blood vessel that can be used to treat different length portions of blood vessel.

To this end, the invention consists in necessaries for treating a blood vessel, of the aforementioned type, characterized in that at least the proximal end of the endoprosthesis is initially outside the ferrule and in that the endoprosthesis initially has a length sufficient to be trimmed to suit the requirements of the vessel to be treated by cutting off and discarding a surplus length of the endoprosthesis at its proximal end.

In particular embodiments of the invention, the necessaries have one or more of the following features:

the support has at its distal end, before the area in which the endoprosthesis is connected to the support, an end-piece for guiding the end of the support in the ferrule for inserting the endoprosthesis therein;

they further include an instrument for cutting off the surplus length at the proximal end of the endoprosthesis;

they include packaging in which the ferrule and the endoprosthesis connected to the support are both packaged; and

the ferrule has, at the end at which the endoprosthesis is inserted, means for constricting the endoprosthesis when it is inserted into the ferrule.

The invention will be better understood after reading the following description which is given by way of example only and with reference to the accompanying drawings, in which:

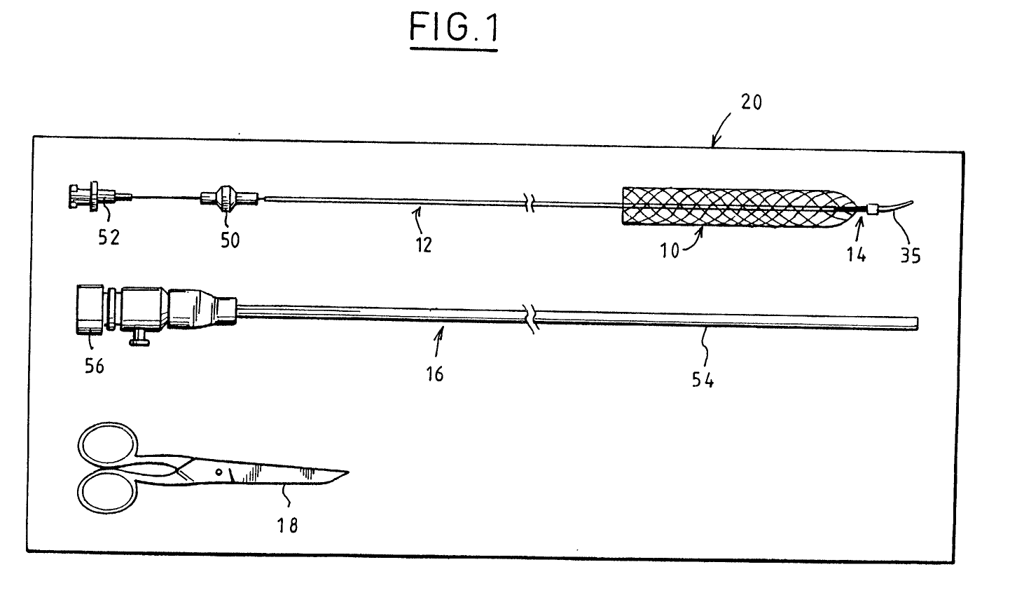

FIG. 1 is an elevation view of the treatment necessaries packaged in common packaging;

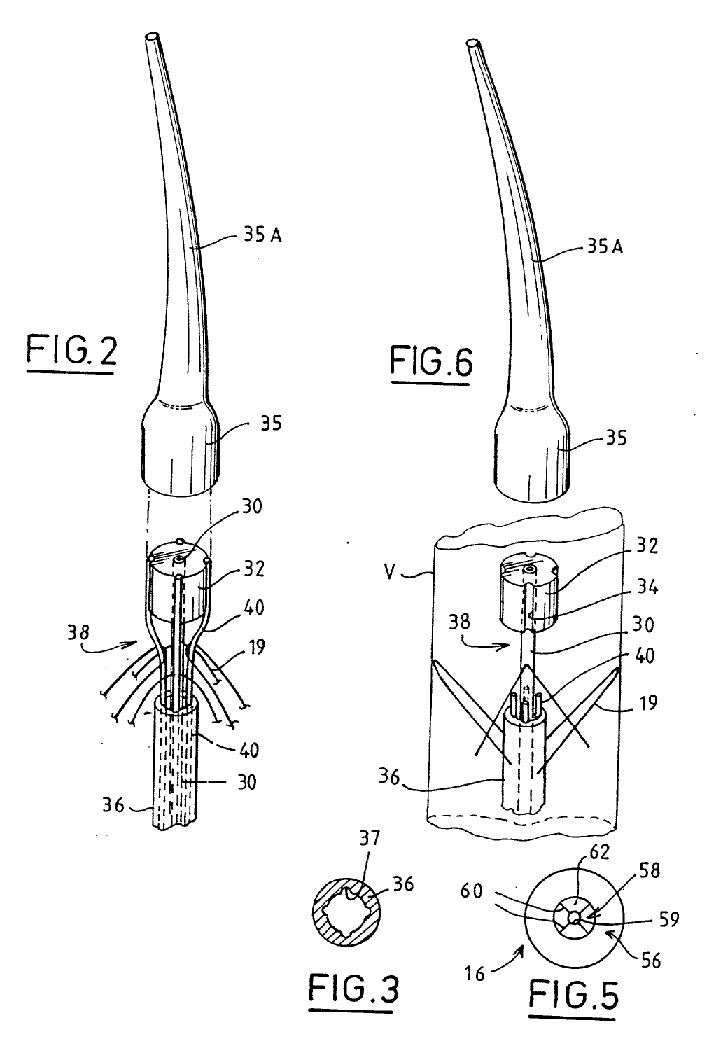

FIG. 2 is an exploded perspective view of the distal end of the support carrying the endoprosthesis before the latter is released;

FIG. 3 is a sectional view of the sheath covering the support of the necessaries from FIG. 1;

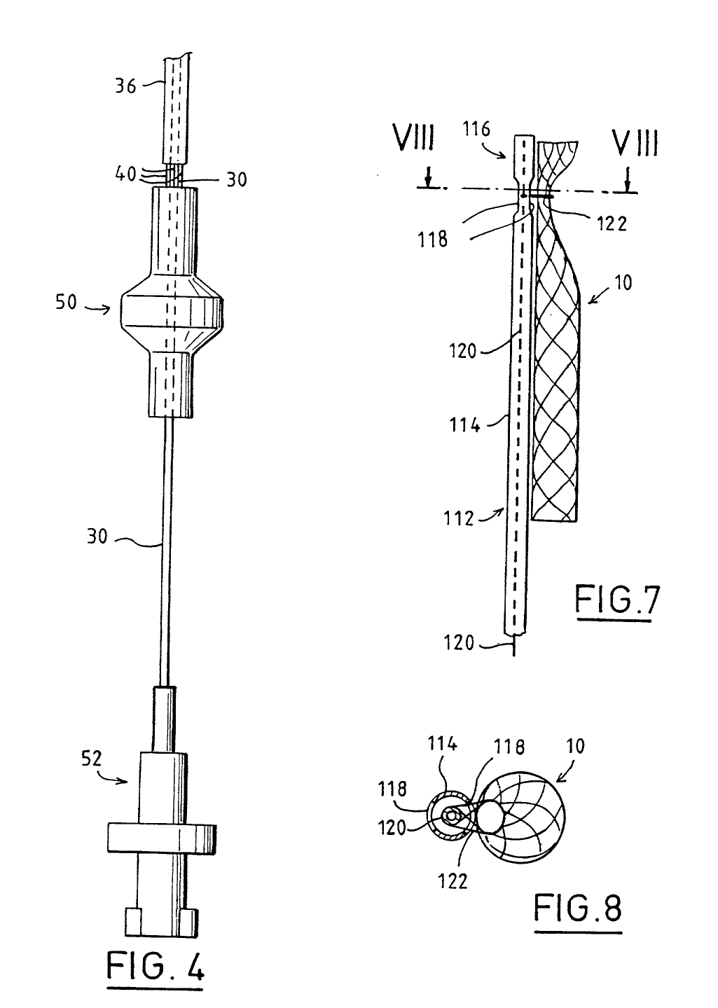

FIG. 4 is an elevation view of actuator means provided at the proximal end of the support;

FIG. 5 is an end view of the proximal end of the ferrule contained in the necessaries;

FIG. 6 is a view analogous to that of FIG. 2 after the end of the endoprosthesis is released inside a blood vessel;

FIG. 7 is a view in elevation of a variant of the distal end of the support, with the tubular endoprosthesis connected to it; and

FIG. 8 is a view in section taken along the line VIII-VIII in FIG. 7 showing the means for releasably retaining the endoprosthesis to the support.

The necessaries shown in FIG. 1 include a tubular endoprosthesis 10 whose distal end is connected to a support 12 by releasable retaining means 14. It further includes a ferrule 16 adapted to guide the endoprosthesis 10 connected to the support 12 when implanting it in a living organism. The necessaries finally include a pair of scissors 18 for cutting the proximal end of the tubular endoprosthesis 10 to trim its length to suit the characteristics of the vessel to be treated.

The three components of the treatment necessaries are packaged in common packaging 20 in the form of a hermetically sealed sachet, for example.

The endoprosthesis comprises a tubular mesh embedded in a film that can stretch and is impermeable to liquids, for example an elastomer. The mesh is made of stainless steel having spring properties so that the endoprosthesis 10 can expand of its own accord. This kind of endoprosthesis is commonly known as a “stent”.

The endoprosthesis is able to deform spontaneously from a compressed state, in which it has a small diameter, to an expanded state, in which it has a larger diameter, this expanded state constituting its unstressed state. This is known per se.

In the embodiment shown, the braid of the endoprosthesis has interlaced looped wires at its distal end, i.e. at the end at which the endoprosthesis is connected to the support 12. Thus, at its distal end, the endoprosthesis has a succession of wires folded back at an angle of substantially 90°. They therefore define loops 19 formed in the main part of the wires constituting the tubular braid. In FIGS. 2 and 6, only the loops 19 of the braid are shown.

The total length of the endoprosthesis packaged in the packaging 20 is greater than that required to treat the patient. In particular, the endoprosthesis has a surplus length at its proximal end, i.e. at the end opposite the end at which it is connected to the support. The surgeon cuts off this surplus before implanting the endoprosthesis.

For example, when packaged, an endoprosthesis having a diameter of 2 cm has a total length of approximately 20 cm. In practice, an endoprosthesis of this diameter when implanted in a blood vessel has a length in the order of 8 cm to 12 cm.

As shown in FIGS. 2 to 4, the support 12 has a core 30 in the form of a flexible metal tube. The inside diameter of the core 30 is suited to threading the core over a filamentary surgical guide installed on the patient prior to implanting the tubular endoprosthesis.

A sleeve 32 is fitted and glued to the distal end of the core 30. The sleeve has four equi-angularly spaced longitudinal grooves 34 on its substantially cylindrical lateral wall.

The sleeve 32 is capped by a flexible end-piece 35 which is extended beyond the sleeve by a flexible skirt 35A whose diameter decreases progressively toward its free end. The latter is open to delimit a passage for the surgical guide. The end-piece 35 constitutes an end guide for inserting the support 12 into the ferrule 16.

The main part of the core 30 is covered by a flexible sheath 36. As shown in FIG. 3, the sheath 36 has a generally circular inside section. It has four equi-angularly spaced longitudinal grooves 37 on its inside surface.

The distal end of the sheath 36 is at a distance from the sleeve 32 such that the sleeve 32 and the sheath 36 delimit an axial notch on the support 12.

As shown in FIG. 2, four filamentary members or strands 40 are mounted so that they can slide in the conduits defined by the grooves 37 between the core 30 and the sheath 36. The strands 40 constitute releasable members for retaining the distal end of the endoprosthesis 10.

The strands 40 are made from a semi-rigid material, i.e. a material capable of elastically following deformation of the core and the sheath but sufficiently rigid to resist transverse traction due to the elasticity of the endoprosthesis 10.

Before the distal end of the endoprosthesis is released, i.e. in the packaged state of the tubular endoprosthesis carried by the support, the distal ends, i.e. the free ends, of the strands 40 are inserted in the longitudinal grooves 34 of the sleeve. Accordingly, in this position, the strands 40 form bridges spanning the notch 38 axially and connecting the distal and proximal portions of the support delimiting the notch.

As shown in FIG. 2, the strands 40 are engaged in the meshes defined by the loops 19 at the distal end of the endoprosthesis 10.

This tightens up this end, the end loops 19 being held against the core 30 by the strands 40 which are in turn held on respective opposite sides of the notch 38 by the sheath 36 and the guide end-piece 35.

The proximal end of the strands 40 projects beyond the sheath 36, as shown in FIG. 4. At this end, the strands 40 are connected to an operating member 50 which slides on a proximal portion of the core 30. The proximal end of the core is provided with a bearing member 52 to facilitate manual sliding of the operating member 50.

The ferrule 16 shown in FIGS. 1 and 5 is for implanting the endoprosthesis in the blood vessel. To this end it includes a flexible tube 54 to hold the endoprosthesis in its compressed state. The tube 54 is open at both ends. At its proximal end it has an end-piece 56 provided with constriction means for radially clamping the endoprosthesis when it is inserted into the ferrule from its proximal end.

The constriction means include an elastic material diaphragm 58, for example. The diaphragm is cut from its central passage 59 by radial cuts 60 delimiting elastically deformable tongues 62 distributed around the periphery of the passage.

The necessaries shown in FIG. 1 are used in the following manner.

When the surgeon has implanted a surgical guide in the area of the vein or artery into which the tubular endoprosthesis is to be implanted, the packaging 20 is opened.

The surgeon then takes the support 12 carrying the endoprosthesis 10. Using the scissors 18, the surgeon cuts off and discards a surplus portion of the proximal end of the tubular endoprosthesis to reduce its length. The surgeon then adjusts the length of the endoprosthesis 10 to suit the needs of the patient. When cutting the proximal portion, the surgeon takes care not to damage the sheath 36 of the support.

The endoprosthesis now being a suitable length, the surgeon inserts the distal end of the support 12 into the ferrule 16. To achieve this, surgeon first inserts the guide end-piece 35. Being made from a flexible material, and having a section that progressively increases to the diameter of the sleeve 32, the end-piece assists with inserting the distal end of the support into the proximal opening of the ferrule 16.

After inserting the end-piece 35, the surgeon continues to push the support 12 into the ferrule 16, progressively compressing the endoprosthesis 10 as it enters the ferrule 16. Radial compression of the endoprosthesis 10 is facilitated by the constriction means at the end of the ferrule.

When the endoprosthesis 10 has been totally inserted into the ferrule 16, the end of the previously installed surgical guide is inserted through the support 12 via the perforated end of the end-piece 35.

The ferrule 16 containing the endoprosthesis 10 connected to the support 12 is then fed along the surgical guide as far as the implantation site for the endoprosthesis 10. This is known per se.

By means of relative sliding movement between the support 12 and the ferrule 16, the surgeon progressively releases the endoprosthesis 10 from the ferrule, via its distal end. The endoprosthesis 10 is then deployed and is pressed against the inside wall of the blood vessel into which it has been implanted.

Only the distal end of the endoprosthesis 10 remains compressed, because it is held by the strands 40.

After the endoprosthesis has been positioned correctly, its distal end is released from the support 12.

To this end, the surgeon causes the strands 40 to slide by moving the operating member 50 toward the proximal end of the guide 30. Upon such sliding motion, the strands 40 escape from the grooves 34 and their free end is drawn back towards the sheath 36. Accordingly, as shown in FIG. 5, the strands 40 are in a position that releases the tubular endoprosthesis. Their free end is then away from the distal portion of the support formed by the sleeve 32 and the end loops 19 of the endoprosthesis are therefore released, because the retaining bridges originally formed by the strands 40 are now interrupted.

The endoprosthesis 10 is therefore pressed entirely against the inside wall of the blood vessel V. The surgical guide, the support 12 and the ferrule 16 are then extracted by pulling them out from their proximal end.

With necessaries of the above kind, in which the endoprosthesis 10 carried by the support 12 is initially outside the ferrule 16, the endoprosthesis having a surplus length or overlength at its proximal end, it is easy for the surgeon to adjust the length of the endoprosthesis during the operation.

The number of items in the range of treatment necessaries is therefore reduced, since only the diameter of the endoprosthesis has to be determined beforehand, the length of the endoprosthesis being adjusted by the surgeon during the operation.

FIG. 7 shows another embodiment of the support 12 and the means 14 for releasably retaining the endoprosthesis 10 at its distal end.

In this embodiment, the support 112 includes a flexible tube 114. Two diametrically opposed openings 118 are formed in its lateral wall, in the vicinity of its distal end 116. A filamentary member 120 slides inside the tube 114. The free end, i.e. the distal end, of the filamentary member 114 extends beyond the notches 118.

In this embodiment, the endoprosthesis 10 is disposed laterally along the support 112 which is outside the conduit defined by the endoprosthesis.

The means for releasably retaining the endoprosthesis 10 include a loop 122 around the distal end of the endoprosthesis 10. The loop is inserted through an opening 118 constituting a notch in the support and is retained there by a filamentary member 120.

As shown in FIG. 8, the loop 122 comprises a flexible strand closed on itself. The resulting loop is flattened and placed around the distal end of the endoprosthesis 10 to cause localized transverse compression thereof. The ends of the loop 122 are superposed to define a closed passage into which the filamentary member 120 is inserted.

The filamentary member 120 is provided at is proximal end with manoeuvring means for sliding it relative to the tube 114.

The endoprosthesis 10 is held without it being possible for the loop 122 to be released because the ends of the loop 122 fitted over the filamentary member 120 are retained in the notch 118 delimited between the distal and proximal portions of the support 112.

The distal end of the endoprosthesis is released after inserting it into a ferrule like the ferrule 16 and placing the latter in a blood vessel.

To this end the filamentary member 120 is slid inside the tube 114 until its distal end is to the rear of the loop 122. This releases the loop 122, enabling deployment of the distal end of the endoprosthesis.

In an embodiment that is not shown, the support 114 includes openings forming longitudinally spaced notches along the entire length of the endoprosthesis. The latter is compressed at each notch by a loop around the endoprosthesis. Each loop is held around the filamentary member 120.

In this embodiment, the endoprosthesis is compressed radially over most of its length, which facilitates inserting it into the ferrule.

The endoprosthesis is released by sliding the filamentary member 120, which progressively releases each retaining loop as its distal end reaches the corresponding notch.

In either embodiment of the means for releasing the prosthesis, the distal end of the endoprosthesis is released by sliding a filamentary member inside the structure of the support. The free end of the filamentary member retains the endoprosthesis as long as the latter is engaged in the distal portion of the support delimiting the notch. Simply by sliding the distal end of the filamentary member, the distal end of the endoprosthesis is free to deploy as soon as the end of the filamentary member has moved away from the distal portion of the support 114.

Accordingly, the endoprosthesis is released simply by sliding a filamentary member, which is very simple to bring about and subject to little risk of dysfunction.

1. Necessaries for treating a blood vessel, of the type including a tubular endoprosthesis (10) whose distal end is connected to a support (12, 112) by releasable retaining means (14) and a ferrule (16) for guiding the endoprosthesis (10) connected to the support (12, 112) for implanting it in a living organism, characterized in that at least the proximal end of the endoprosthesis is initially outside the ferrule (16) and in that the endoprosthesis (10) initially has a length sufficient to be trimmed to suit the requirements of the vessel to be treated by cutting off and discarding a surplus length of the endoprosthesis (10) at its proximal end.

2. Necessaries according to claim 1 characterized in that the support (12) has at its distal end, before the area in which the endoprosthesis (10) is connected to the support (12), an end-piece (35) for guiding the end of the support in the ferrule (16) for inserting the endoprosthesis (10) therein.

3. Necessaries according to claim 1 or claim 2 characterized in that they further include an instrument (18) for cutting off the surplus length at the proximal end of the endoprosthesis (10).

4. Necessaries according to any one of the preceding claims characterized in that they include packaging (20) in which the ferrule (16) and the endoprosthesis (10) connected to the support (12) are both packaged.

5. Necessaries according to any one of the preceding claims characterized in that the ferrule (16) has, at the end at which the endoprosthesis (10) is inserted, means (54) for constricting the endoprosthesis (10) when it is inserted into the ferrule (16).
